(19) United States
(12) Patent Application Publication
(10) Pub. No.: US 2022/0198540 A1
(43) Pub. Date: Jun. 23, 2022

(54) INFORMATION PROVIDING SYSTEM

(71) Applicant: TOYOTA JIDOSHA KABUSHIKI KAISHA, Toyota-shi (JP)

(72) Inventors: Hideyuki SAKURAI, Toyota-shi (JP); Nobuhiro NAKANO, Nagakute-shi (JP); Kazuhisa MAEDA, Toyota-shi (JP); Yohei TANIGAWA, Toyota-shi (JP); Keisuke ITOU, Tokyo (JP); Kantarou SHINMA, Tokyo (JP); Nobuki HAYASHI, Nisshin-shi (JP); Tetsuro SAKAGUCHI, Toyota-shi (JP); Yukari OGAWA, Obu-shi (JP)

(73) Assignee: TOYOTA JIDOSHA KABUSHIKI KAISHA, Toyota-shi (JP)

(21) Appl. No.: 17/552,299

(22) Filed: Dec. 15, 2021

(30) Foreign Application Priority Data
Dec. 18, 2020 (JP) 2020-209823

(51) Int. Cl. G06Q 30/06; G06Q 30/02; G06Q 50/12; G06Q 10/02; G06F 16/538; G06V 20/68

(52) U.S. Cl. CPC G06Q 30/0623; G06Q 30/0207; G06Q 30/0641; G06V 20/68; G06Q 10/02; G06F 16/538; G06Q 50/12

(57) ABSTRACT

An information providing system includes a first portable terminal of a first visitor who visits a theme park and a server that communicates with the first portable terminal, in which the information providing system provides information on food provided in the theme park, the server includes a database in which an image of the food is associated with the information on the food, and when the first visitor takes a picture of food eaten by a second visitor and uploads the taken picture, the server specifies the food in the picture with reference to the database, and transmits the information on the specified food to the first portable terminal of the first visitor.

### CROSS-REFERENCE TO RELATED APPLICATION

This application claims priority to Japanese Patent Application No. 2020-209823 filed on Dec. 18, 2020, incorporated herein by reference in its entirety.

### BACKGROUND

### 1. Technical Field

The present disclosure relates to an information providing system that provides information on food or products provided in a predetermined area to a visitor who visits the predetermined area.

### 2. Description of Related Art

A method of recognizing food based on an image captured by a camera and performing various processes based on a result of recognition has been proposed. For example, Japanese Unexamined Patent Application Publication No. 2020-42401 (JP 2020-42401 A) proposes a method of recognizing whether or not an image captured by a camera is food, and in a case where the image is recognized as the food, displaying calorie information of the food.

### SUMMARY

Incidentally, various tourist facilities have been constructed recently. In a predetermined area, such as the tourist facility, it is requested for a visitor to have a good time and repeatedly visit the tourist facility many times.

In some cases, when the visitor walks in the predetermined area, the visitor sees delicious food (including drink) eaten by another visitor. Similarly, in some cases, when the visitor walks in the predetermined area, the visitor sees a nice product owned by another visitor. In these cases, the visitor often wonders what the food or the product is, and desires to immediately acquire information on the food or products, such as a name, a provision place in the predetermined area, and a provision price. However, the visitor often cannot ask other strange visitors directly, and thus it is not easy to acquire such information.

Therefore, the present disclosure enables the visitor who visits the predetermined area to easily acquire the information on the food eaten by another visitor or the product owned by another visitor.

An aspect of the present disclosure relates to an information providing system including a portable terminal of a visitor who visits a predetermined area, and a server. The server is configured to communicate with the portable terminal. The information providing system provides information on a plurality of foods provided in the predetermined area or a plurality of products provided in the predetermined area. The portable terminal of the visitor is configured to take a picture and upload the taken picture to the server. The server includes a database in which images of the foods provided in the predetermined area or the products provided in the predetermined area are associated with the information on the foods or the products. The server is configured to, when the visitor takes a picture of one food eaten by another visitor or one product owned by the other visitor and uploads the taken picture to the server, specify the one food or the one product in the uploaded picture with reference to the database, and transmit the information on the specified one food or the specified one product to the portable terminal of the visitor.

As a result, the visitor can easily acquire the information on the food or the product solely by taking the picture of the food eaten by the other visitor or the product owned by the other visitor and uploading the taken picture.

In the information providing system according to the aspect of the present disclosure, the information may be a name, a provision place, and a provision price of the specified one food or the specified one product, and the server may be configured to transmit the information and the image of the specified one food or the specified one product stored in the database to the portable terminal of the visitor.

As described above, the server transmits the name, the provision place, the provision price, and the image of the one food eaten by the other visitor or the one product owned by the other visitor, so that the visitor can go buy the food or the product immediately.

The information providing system according the aspect of the present disclosure may further include another portable terminal of the other visitor, the other portable terminal being configured to communicate with the server, in which the server is configured to, when the visitor places an order for the one food eaten by the other visitor or the one product owned by the other visitor based on the information transmitted to the portable terminal, give a point to the other portable terminal of the other visitor.

By giving the point to the other visitor as described above, a repeat rate of the other visitor can be increased. In addition, giving the point motivates the other visitor to actively advertise the food or the product, and the other visitor serves as an advertising body for the food or the product in the predetermined area. As a result, the repeat rate of the visitor can be increased.

In the information providing system according to the aspect of the present disclosure, a one-tap button may be displayed on a display of the portable terminal when the portable terminal of the visitor takes the picture of the one food or the one product, the picture may be uploaded to the server when the visitor presses the one-tap button, and the server may be configured to specify the one food or the one product in the uploaded picture with reference to the database, and make a reservation for the specified one food or a reservation for the specified one product.

As a result, the visitor can easily make a reservation for the one food eaten by the other visitor or the one product owned by the other visitor by a simple method.

The present disclosure enables the visitor who visits the predetermined area to easily acquire the information on the food eaten by the other visitor or the product owned by the other visitor.

### BRIEF DESCRIPTION OF THE DRAWINGS

Features, advantages, and technical and industrial significance of exemplary embodiments of the disclosure will be described below with reference to the accompanying drawings, in which like signs denote like elements, and wherein: FIG. 1 is a system diagram showing a configuration of an information providing system according to an embodiment;

FIG. 2 is a flowchart showing an operation of the information providing system according to the embodiment; and

FIG. 3 is a flowchart showing another operation of the information providing system according to the embodiment.

### DETAILED DESCRIPTION OF EMBODIMENTS

Hereinafter, a case where an information providing system 100 is applied to a theme park 80 that is a predetermined area will be described with reference to the drawings. As shown in FIG. 1, the information providing system 100 includes a first portable terminal 12 of a first visitor 10 of the theme park 80, a second portable terminal 22 that is another portable terminal of a second visitor 20 who is another visitor different from the first visitor 10, a server 40, a food selling store 50, and a product selling store 55.

Here, the theme park 80 is a tourist facility produced based on a specific theme, such as culture, a country, a story, a movie, or an era, as a whole. In the theme park 80, a facility that produces a scene of a specific movie or the like and performs an attraction making the visitor feel as if the visitor is with a character appearing in the movie is provided. Further, in the theme park 80, the food selling store 50 that provides a plurality of foods 52 or the product selling store 55 that sells a plurality of products 57, such as a souvenir with the character, is provided.

The first and second visitors 10, 20 own the first and second portable terminals 12, 22, respectively. The first and second portable terminals 12, 22 each communicate with the server 40 via a communication line 30, such as the Internet or a telephone line. The first and second portable terminals 12, 22 each have a built-in camera that takes a picture.

The server 40 is a computer internally including a CPU 42 as a processor that performs information processing, and a memory 44 that stores a program, operation data, or the like. A database 46 is stored in the memory 44.

The memory 44 stores the database 46 in which images of the foods 52 provided in the theme park 80 or the products 57 provided in the theme park 80 are stored in association with the information of the food 52 or the product 57. Here, the information includes a name, a provision place, and a provision price of the food 52 or the product 57.

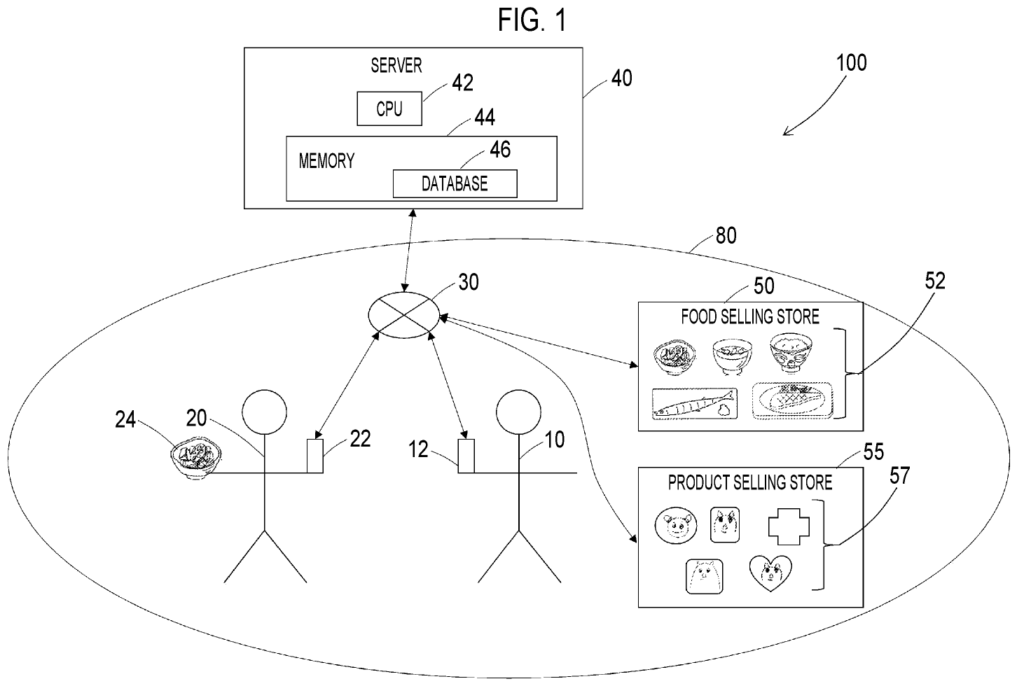

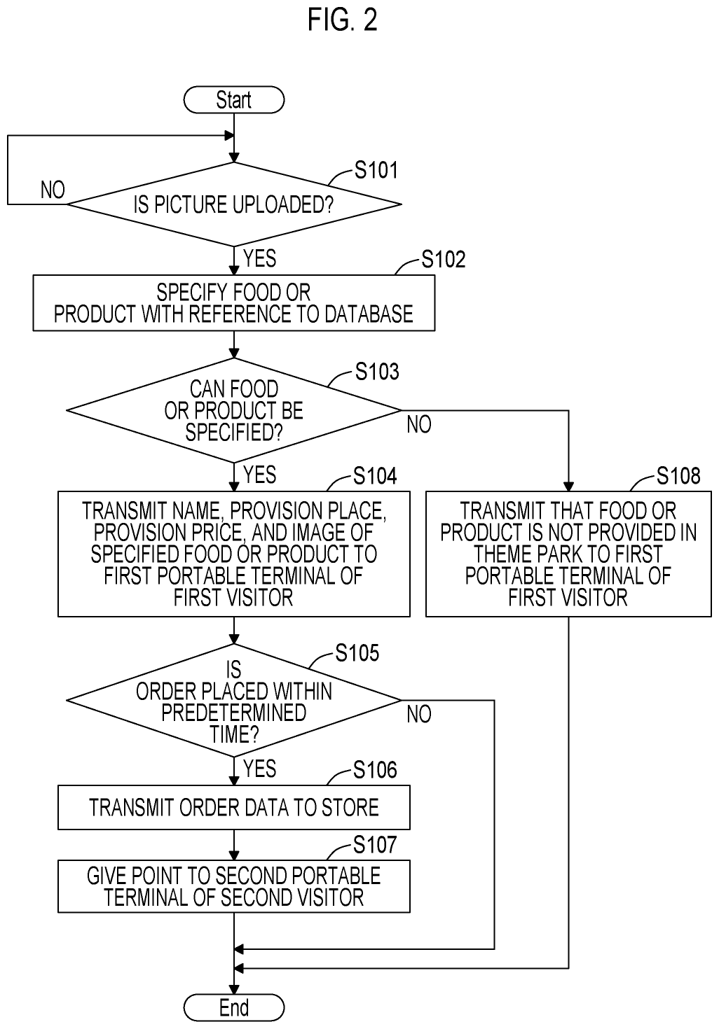

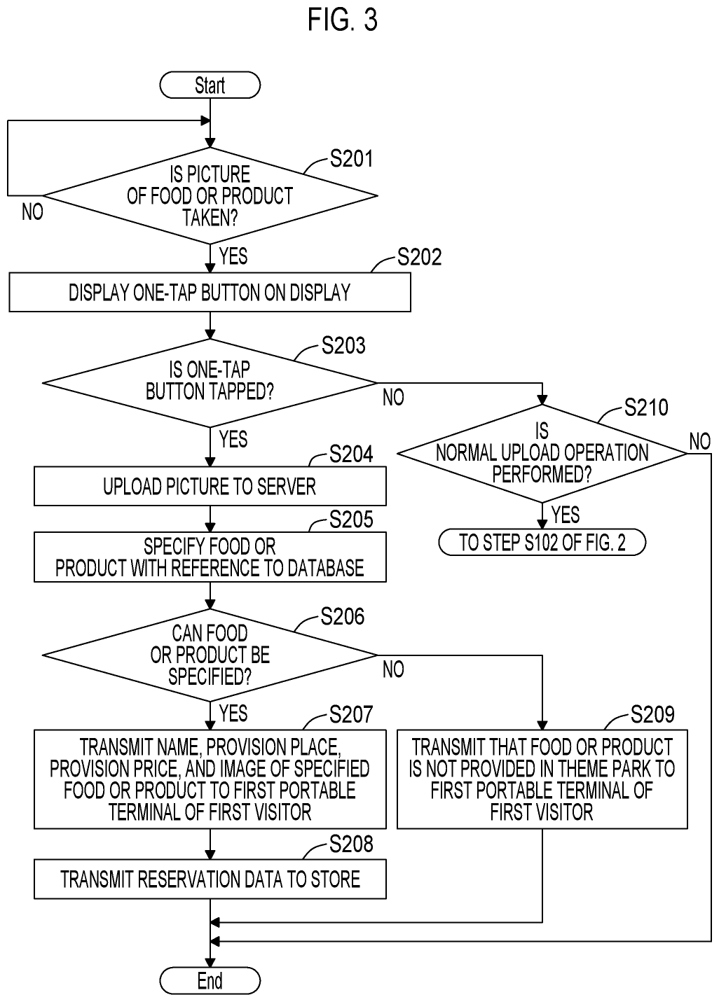

Next, an operation of the information providing system 100 configured as described above will be described with reference to FIG. 2. In the following, description will be made on the assumption that while the second visitor 20 eats one food 24 in the theme park 80, the first visitor 10 passes and feels that the food 24 eaten by the second visitor 20 looks delicious.

The first visitor 10 asks the second visitor 20 whether or not the first visitor 10 can take a picture of the food 24, and after obtaining understanding of the second visitor 20, the first visitor 10 takes a picture of the food 24 by the first portable terminal 12 and uploads the taken picture to the server 40.

As shown in step S101 of FIG. 2, the server 40 waits until the picture is uploaded, and when the picture is uploaded, determines YES in step S101 of FIG. 2 and proceeds to step S102 of FIG. 2. The server 40 compares the images of the foods 52 stored in the database 46 with the uploaded picture in step S102 of FIG. 2, and in a case where the image and the picture match, the server 40 specifies that the food taken in the uploaded picture is the food 24 corresponding to the image. When the server 40 can specify the food taken in the picture, the server 40 determines YES in step S103 of FIG. 2 and proceeds to step S104 of FIG. 2.

In step S104 of FIG. 2, the server 40 reads out the name, the provision place, and the provision price of the food 24 stored in the database 46 in association with the image from the database 46. Then, the server 40 transmits the readout name, provision place, and provision price of the food 24, and the image of the food 24 stored in the database 46 to the first portable terminal 12 of the first visitor 10.

The transmitted name, provision place, provision price, and image of the food 24 are displayed on a display of the first portable terminal 12. As a result, the first visitor 10 can confirm the information and the image of the food 24 eaten by the second visitor 20, and can place an order for the food 24 by the first portable terminal 12.

When the first visitor 10 performs an operation of placing the order for the food 24 based on the information on the food 24 transmitted from the server 40, an order request is transmitted from the first portable terminal 12 to the server 40.

As shown in step S105 of FIG. 2, when the server 40 receives the order request from the first portable terminal 12 of the first visitor 10 within predetermined time, the server 40 determines YES in step S105 of FIG. 2 and proceeds to step S106 of FIG. 2. In step S106 of FIG. 2, the server 40 first transmits order data to the food selling store 50 that provides the food 24 read out from the database 46.

When the order is received, the food selling store 50 that receives the order data cooks and prepares the ordered food 24, and when the first visitor 10 visits the food selling store 50, the food 24 is sold to the first visitor 10.

Further, in step S107 of FIG. 2, the server 40 gives the point that can be used in the theme park 80 to the second portable terminal 22 of the second visitor 20 who eats the food 24 and permits the first visitor 10 to take the picture. The second visitor 20 can use the point to enter the attraction in the theme park 80 and purchase the product 57, such as a souvenir.

On the other hand, in a case where the order is not placed within the predetermined time after the server 40 transmits the information and the image of the food 24 to the first portable terminal 12 of the first visitor 10, the server 40 determines NO in step S105 of FIG. 2, skips steps 5106, 5107 of FIG. 2, and terminates the process.

In addition, in a case where there is no image stored in the database 46 that matches the picture in step S102 of FIG. 2, the server 40 determines NO in step S103 of

FIG. 2 and proceeds to step S108 of FIG. 2. Then, the server 40 transmits a message that the food in the picture is not provided in the theme park 80 to the first portable terminal 12 of the first visitor 10.

In the above description, the operation of the information providing system 100 in a case where the first visitor 10 takes the picture of one food 24 eaten by the second visitor 20 and uploads the taken picture has been described. However, an operation of the information providing system 100 in a case where the first visitor 10 takes the picture of one product owned by the second visitor 20 and related to the theme park 80 and uploads the taken picture is the same as the operation in a case where the picture of the food 24 is taken and uploaded except that a transmission destination of the order data from the server 40 is the product selling store 55.

As described above, with the information providing system 100 according to the embodiment, the first visitor 10 can easily purchase the food 24 or the product solely by taking and uploading the picture of the food 24 eaten by the second visitor 20 or the product owned by the second visitor 20 to receive the name, the provision place, the provision price, and the image of the food 24 or the product.

In addition, in a case where the first visitor 10 places the order for the food 24 or the product based on the information on the food 24 or the product received from the server 40, the server 40 gives the point that can be used in the theme park 80 to the second portable terminal 22 of the second visitor 20 who permits taking the picture of the food 24 or the product, so that the repeat rate of the second visitor 20 can be increased. In addition, giving the point motivates the second visitor 20 to actively advertise the food 52 or the product 57, and the second visitor 20 serves as an advertising body for the food 52 or the product 57 in the theme park 80. As a result, the repeat rate of the first visitor 10 can be increased.

Next, another operation of the information providing system 100 will be described with reference to FIG. 3. An icon for performing a specific operation is displayed on the display of the first portable terminal 12 of the first visitor 10. When the first visitor 10 clicks or taps the icon, a taking mode is set in which the first visitor 10 takes the picture of the food 24 eaten by the second visitor 20. Then, when the first visitor 10 takes the picture of the food 24 of the second visitor 20 by the first portable terminal 12, the first portable terminal 12 determines YES in step S201 of FIG. 3, proceeds to step S202 of FIG. 2 to display a one-tap button on the display. The one-tap button may be, for example, a button displaying the characters “Like”.

Then, when the first visitor 10 taps the one-tap button, the first portable terminal 12 determines YES in step S203 of FIG. 3, proceeds to step S204 of FIG. 3, and uploads the picture of the food 24 to the server 40. The server 40 receives the picture and specifies the food 24 with reference to the database 46 in step S205 of FIG. 3 in the same manner as in step S102 shown in FIG. 2 of the previous operation. Then, when the food 24 can be specified, the server 40 determines YES in step S206 of FIG. 3, proceeds to step S207 of FIG. 3, and transmits the information and the image of the specified food 24 to the first portable terminal 12 in the same manner as in step S104 of FIG. 2 of the operation described above.

Then, the server 40 proceeds to step S208 of FIG. 3 and transmits reservation data of the food 24 to the food selling store 50 that provides the food 24.

On the other hand, in a case where the food 24 cannot be specified in step S205 of FIG. 3, the server 40 determines NO in step S206 of FIG. 3 and transmits the message that the food in the picture is not provided in the theme park 80 to the first portable terminal 12 of the first visitor 10 in step S209 of FIG. 3 in the same manner as in step S108 of FIG. 2 of the operation described above.

In addition, in a case where the first visitor 10 performs a normal upload operation without pressing the one-tap button, the first portable terminal 12 determines NO in step S203 of FIG. 3 and proceeds to step S210 of FIG. 3. Then, the first portable terminal 12 determines YES in step S210 of FIG. 3, and proceeds to step S102 of FIG. 2. Then, by executing steps S102 to S107 of FIG. 2, the server 40 specifies the food 24, and in a case where the food 24 can be specified, the server 40 transmits the information and the image of the food 24 to the first portable terminal 12. In addition, in a case where an order signal is input from the first portable terminal 12, the server 40 transmits the order data to the food selling store 50.

On the other hand, in a case where the one-tap button is not pressed and the normal upload operation is also not performed within the predetermined time, the first portable terminal 12 determines NO in both steps S203 and S210 of FIG. 3 and terminates the operation without uploading the picture.

In the same manner as in the operation described above, the operation of the information providing system 100 in a case where the first visitor 10 takes the picture of the product owned by the second visitor 20 and related to the theme park 80 is the same as the operation as in a case where the picture of the food 24 is taken except that a transmission destination of the reservation data from the server 40 is the product selling store 55.

In the other operation described above, the first visitor 10 can automatically upload the picture and make the reservation for the food 24 in the taken picture solely by pressing the one-tap button displayed on the display of the first portable terminal 12, and thus the first visitor 10 can make the reservation for the food 24 eaten by the second visitor 20 by a simple method. Here, the reservation may be a reservation for tomorrow's lunch, for example.

A case where the information providing system 100 is applied to the theme park 80 that is the predetermined area has been described above, but the information providing system 100 can also be applied to an area other than the theme park 80. For example, the information providing system 100 may be applied to a sports facility, such as a baseball stadium or a soccer field in which various products related to a professional player or team are sold and drinks and the like are sold during competition watching. Further, the information providing system 100 may be applied to an event venue in which a product related to an event is sold or the food is sold.

What is claimed is:

1. An information providing system comprising:
a portable terminal of a visitor who visits a predetermined area; and
a server configured to communicate with the portable terminal, wherein:
the information providing system provides information on a plurality of foods provided in the predetermined area or a plurality of products provided in the predetermined area;
the portable terminal of the visitor is configured to take a picture and upload the taken picture to the server;
the server includes a database in which images of the foods provided in the predetermined area or the products provided in the predetermined area are associated with the information on the foods or the products; and
the server is configured to, when the visitor takes a picture of one food eaten by another visitor or one product owned by the other visitor and uploads the taken picture to the server, specify the one food or the one product in the uploaded picture with reference to the database, and transmit the information on the specified one food or the specified one product to the portable terminal of the visitor.

2. The information providing system according to claim 1, wherein:
the information is a name, a provision place, and a provision price of the specified one food or the specified one product; and
the server is configured to transmit the information and the image of the specified one food or the specified one product stored in the database to the portable terminal of the visitor.

3. The information providing system according to claim 1, further comprising another portable terminal of the other visitor, the other portable terminal being configured to communicate with the server,
wherein the server is configured to, when the visitor places an order for the one food eaten by the other visitor or the one product owned by the other visitor based on the information transmitted to the portable terminal, give a point to the other portable terminal of the other visitor.

4. The information providing system according to claim 1, wherein:
a one-tap button is displayed on a display of the portable terminal when the portable terminal of the visitor takes the picture of the one food or the one product;
the picture is uploaded to the server when the visitor presses the one-tap button; and
the server is configured to specify the one food or the one product in the uploaded picture with reference to the database, and make a reservation for the specified one food or a reservation for the specified one product.
